SEQUENCE LISTING (18 sequences)

SEQ ID NO: 1 - protein, 5 aa, Artificial Sequence
  source: /note=“Description of Artificial Sequence Synthetic peptide”
KAWMS

SEQ ID NO: 2 - protein, 19 aa, Artificial Sequence
  source: /note=“Description of Artificial Sequence Synthetic peptide”
RIKSTADGGTTSYAAPVEG

SEQ ID NO: 3 - protein, 2 aa, Artificial Sequence
  source: /note=“Description of Artificial Sequence Synthetic peptide”
AH

SEQ ID NO: 4 - protein, 11 aa, Artificial Sequence
  source: /note=“Description of Artificial Sequence Synthetic peptide”
SGEALPMQFAH

SEQ ID NO: 5 - protein, 7 aa, Artificial Sequence
  source: /note=“Description of Artificial Sequence Synthetic peptide”
KDSERPS

SEQ ID NO: 6 - protein, 11 aa, Artificial Sequence
  source: /note=“Description of Artificial Sequence Synthetic peptide”
QSPDSTNTYEV

SEQ ID NO: 7 - protein, 10 aa, Artificial Sequence
  source: /note=“Description of Artificial Sequence Synthetic peptide”
GFDFEKAWMS

SEQ ID NO: 8 - protein, 113 aa, Artificial Sequence
  source: /note=“Description of Artificial Sequence Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSS

SEQ ID NO: 9 - protein, 108 aa, Artificial Sequence
  source: /note=“Description of Artificial Sequence Synthetic polypeptide”
SYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVL

SEQ ID NO: 10 - protein, 442 aa, Artificial Sequence
  source: /note=“Description of Artificial Sequence Synthetic polypeptide”
EVQLVESGGGLVEPGGSLRLSCAVSGFDFEKAWMSWVRQAPGQGLQWVARIKSTADGGTTSYAAPVEGRFIISRDDSRNMLYLQMNSLKTEDTAVYYCTSAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, Artificial Sequence
  source: /note=“Description of Artificial Sequence Synthetic polypeptide”
SYELTQPPSVSVSPGQTARITCSGEALPMQFAHWYQQRPGKAPVIVVYKDSERPSGVPERFSGSSSGTTATLTITGVQAEDEADYYCQSPDSTNTYEVFGGGTKLTVLSQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 13 - protein, 12 aa, Homo sapiens
FMKGLSKAKEGV

SEQ ID NO: 14 - protein, 7 aa, Homo sapiens
FMKGLSK

SEQ ID NO: 15 - protein, 22 aa, Artificial Sequence
  source: /note=“Description of Artificial Sequence Synthetic peptide”
MDMRVPAQLLGLLLLWFPGSRC

SEQ ID NO: 16 - protein, 22 aa, Artificial Sequence
  source: /note=“Description of Artificial Sequence Synthetic peptide”
MDMRVPAQLLGLLLLWLPGARC

SEQ ID NO: 17 - DNA, 711 bp, Artificial Sequence
  source: /note=“Description of Artificial Sequence Synthetic polynucleotide”
atggacatgcgggtgcccgcccagctgctgggcctgctgctgctgtggctccctggcgccagatgtagctacgagctgacccagccccccagcgtcagcgtcagccccggccagaccgccaggatcacctgcagcggcgaggccctgcccatgcagttcgcccactggtaccaacagaggccaggcaaggccccagtgatcgtggtgtacaaagacagtgagagaccctcaggtgtccctgagcgattctctggctcctcttccgggacaacagccaccttgaccatcactggagtccaggcagaagatgaggctgactattactgccagtctccagacagcactaacacttatgaagtcttcggcggagggaccaagctgaccgtcctgagtcagcccaaggctgccccctccgtcactctgttccctccctcctccgaggaacttcaagccaacaaggccacactggtctgtctcatcagtgacttctaccctggagccgtgacagtggcctggaaggcagatagcagccccgtcaaggctggagtggagaccaccacaccctccaaacaaagcaacaacaaatacgctgccagcagctacctgagcctgacacctgagcagtggaagtcccacagaagctacagctgccaggtcacccatgaagggagcaccgtggagaagacagtggcccctacagaatgttcatag

SEQ ID NO: 18 - DNA, 1395 bp, Artificial Sequence
  source: /note=“Description of Artificial Sequence Synthetic polynucleotide”
atggacatgcgggtgcccgcccagctgctgggcctgctgctgctgtggttccccggctctcggtgcgaggtgcagctggtggagtccgggggaggtctggtcgagcctggggggtccctgagactctcctgtgcagtctccggattcgatttcgaaaaagcctggatgagttgggtccgccaggctccagggcaggggctgcagtgggttgcccggatcaagagcacagctgatggtgggacaacaagctacgccgcccccgtggaaggcagattcatcatctcaagagatgattccagaaacatgctttatctgcaaatgaacagtctgaaaactgaagacacagccgtctattattgtacatcagcccactggggccagggaaccctggtcaccgtctcctctgcctccaccaagggcccatccgtcttccctctggcaccctcctccaaaagcacctctgggggcacagccgccctgggctgcctggtcaaggactacttccccgaacctgtgaccgtctcctggaactcaggcgccctgaccagcggcgtgcacaccttccctgctgtcctgcaatcctccggactctactccctctcttccgtggtgaccgtgccctccagcagcttgggcacccagacctacatctgcaacgtgaatcacaagcccagcaacaccaaggtggacaagagagttgagcccaaatcttgtgacaaaactcacacatgcccaccctgcccagcacctgaactcctggggggaccctcagtcttcctcttccccccaaaacccaaggacaccctcatgatctcccggacccctgaggtcacatgcgtggtggtggacgtgagccacgaagaccctgaggtcaagttcaactggtatgttgacggcgtggaggtccataatgccaagacaaagcctcgggaggagcagtacaacagcacctaccgcgtggtcagcgtcctcaccgtcctgcaccaagactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagcctgagaacaactacaagaccacacctcccgtgctggactccgacggctccttcttcctctattccaaactcaccgtggacaagagcaggtggcagcaggggaacgtcttctcatgctccgtgatgcatgaggctctgcacaaccactacacccagaagagcctctccctgtcccctggttga

(19) United States
(12) Patent Application Publication
(10) Pub. No.: US 2020/0377579 A1
(43) Pub. Date: Dec. 3, 2020

(54) COMPOSITIONS AND METHODS FOR TREATING SYNUCLEINOPATHIES

(71) Applicant: Biogen International Neuroscience GmbH, Baar (CH)

(72) Inventors: Natasha Penner, Newton, MA (US); Kumar Kandadi Muralidharan, Burlington, MA (US)

(21) Appl. No.: 16/497,055

(22) PCT Filed: Mar. 30, 2018
(86) PCT No.: PCT/IB2018/052236

§ 371 Date: Sep. 24, 2019

Related U.S. Application Data
(60) Provisional application No. 62/528,790, filed on Jul. 5, 2017.
(60) Provisional application No. 62/479,818, filed on Mar. 31, 2017.

(51) Int. Cl. C07K 16/18

(52) U.S. Cl. CPC C07K 16/18; C07K 2317/565; A61K 2039/505; C07K 2317/92; C07K 2317/21

(57) ABSTRACT

Dosage regimens of anti-α-synuclein antibodies are provided. These dosage regimens find use in the treatment of synucleinopathies such as Parkinson's disease (PD), Parkinson's Disease Dementia (PDD), dementia with Lewy bodies (DLB), Lewy body variant of Alzheimer's disease (LBVAD), pure autonomic failure (PAF), multiple system atrophy (MSA), and neurodegeneration with brain iron accumulation type-1 (NBIA-I).

### CROSS-REFERENCE TO RELATED APPLICATIONS

This application claims the benefit of priority of U.S. Provisional Appl. No. 62/479,818, filed Mar. 31, 2017, and U.S. Provisional Appl. No. 62/528,790, filed July 5, 2017, the content of both of which are incorporated by reference herein in their entirety.

### FIELD

The present application relates generally to dosage regimens for the clinical use of anti-α-synuclein antibodies.

### BACKGROUND

Protein misfolding and aggregation are pathological aspects of numerous neurodegenerative diseases (e.g., synucleinopathies). Aggregates of α-synuclein are major components of the Lewy bodies and Lewy neurites associated with Parkinson's disease (PD). A natively unfolded protein, α-synuclein can adopt different aggregated morphologies, including oligomers, protofibrils and fibrils. The small oligomeric aggregates have been shown to be particularly toxic.

In order to treat the growing numbers of patients with synucleinopathies, there is a need for a therapeutic antibody against α-synuclein and appropriate dosage regimens for the clinical use of such an anti-α-synuclein antibody.

### SUMMARY

This disclosure relates, in part, to dosage regimens of α-synuclein antibodies or α-synuclein-binding fragments thereof and their use in the treatment of a synucleinopathy.

In one aspect, provided is a method of treating a synucleinopathy in a human subject in need thereof The method involves administering intravenously to the human subject an anti-α-synuclein antibody at a dose of 3 mg per kg, 5 mg per kg, 15 mg per kg, 45 mg per kg, 90 mg per kg, or 135 mg per kg of body weight of the human subject. The anti-α-synuclein antibody comprises an immunoglobulin heavy chain variable region (VH) and an immunoglobulin light chain variable region (VL), wherein: the VH comprises VH complementarity determining regions (VH-CDRs), wherein: VH-CDR1 consists of the amino acid residues of SEQ ID NO:1; VH-CDR2 consists of the amino acid residues of SEQ ID NO:2; and VH-CDR3 consists of the amino acid residues of SEQ ID NO:3; and the VL comprises VL-CDRs, wherein: VL-CDR1 consists of the amino acid residues of SEQ ID NO:4; VL-CDR2 consists of the amino acid residues of SEQ ID NO:5; and VL-CDR3 consists of the amino acid residues of SEQ ID NO:6.

In some embodiments of this aspect, the synucleinopathy is Parkinson's disease (PD), Parkinson's disease dementia (PDD), dementia with Lewy bodies (DLB), the Lewy body variant of Alzheimer's disease (LBVAD), multiple systems atrophy (MSA), pure autonomic failure (PAF), or neurodegeneration with brain iron accumulation type-1 (NBIA-I). In a particular embodiment, the synucleinopathy is Parkinson's disease (PD). In some cases, the PD is mild PD. In other instances, the PD is moderate PD.

In another aspect, this disclosure features a method of treating abnormal accumulation or deposition of α-synuclein in the central nervous system in a human subject in need thereof. The method includes administering intravenously to the human subject an anti-α-synuclein antibody at a dose of 3 mg per kg, 5 mg per kg, 15 mg per kg, 45 mg per kg, 90 mg per kg, or 135 mg per kg of body weight of the human subject. The anti-α-synuclein antibody comprises an immunoglobulin heavy chain variable region (VH) and an immunoglobulin light chain variable region (VL), wherein: the VH comprises VH complementarity determining regions (VH-CDRs), wherein: VH-CDR1 consists of the amino acid residues of SEQ ID NO:1; VH-CDR2 consists of the amino acid residues of SEQ ID NO:2; and VH-CDR3 consists of the amino acid residues of SEQ ID NO:3; and the VL comprises VL-CDRs, wherein: VL-CDR1 consists of the amino acid residues of SEQ ID NO:4; VL-CDR2 consists of the amino acid residues of SEQ ID NO:5; and VL-CDR3 consists of the amino acid residues of SEQ ID NO:6.

In some embodiments of this aspect, the human subject has been identified as having abnormal accumulation or deposition of α-synuclein in the central nervous system. In certain cases, the human subject is identified by in vivo imaging of α-synuclein (e.g., in the brain) by a method comprising positron emission tomography (PET), single photon emission tomography (SPECT), near infrared (NIR) optical imaging, magnetic resonance imaging (MRI), dopamine transporter imaging, or substantia nigra ultrasonography. In other cases, the human subject is identified by assaying the level of α-synuclein in a blood, plasma, or cerebrospinal fluid (CSF) sample obtained from the subject following peripheral administration to the subject of the anti-α-synuclein antibody and comparing the assayed level of α-synuclein in the subject to a reference standard, wherein the difference or similarity between the level of α-synuclein in the blood, plasma, or CSF sample and the reference standard correlates with the level of α-synuclein in the brain of the subject. In some embodiments, the human subject has been identified by having symptoms of a synucleinopathy.

In some embodiments of this aspect, the human subject is at risk of developing Parkinson's disease (e.g., due to the subject having a genetic risk factor such as a mutation in the SNCA, LRRK2, Parkin, PINK1, DJ1, ATP13A2, PLA2G6, FBXO7, UCHL1, GIGYF2, HTRA2, or EIF4G1 gene) or the human subject has prodromal Parkinson's disease (e.g., the subject has symptoms or clusters of symptoms associated with future development of Parkinson's disease such as hyposmia, REM Behavior Disorder, seborrheic dermatosis, and/or certain autonomic symptoms including but not limited to orthostatic hypotension, impotence in males, and/or disorders of bladder control).

These embodiments apply to both the above-described aspects. In certain embodiments, the VH consists of the amino acid sequence set forth in SEQ ID NO:8. In certain embodiments, the VL consists of the amino acid sequence set forth in SEQ ID NO:9. In certain embodiments, the VH consists of the amino acid sequence set forth in SEQ ID NO:8 and the VL consists of the amino acid sequence set forth in SEQ ID NO:9. In certain embodiments, the antibody comprises a human IgG1 heavy chain constant region. In some embodiments, the antibody comprises a human lambda light chain constant region. In certain embodiments, the antibody comprises a human IgG1 heavy chain constant region and a human lambda light chain constant region. In yet other embodiments, the antibody comprises a heavy chain and a light chain, wherein the heavy chain consists of the amino acid sequence set forth in SEQ ID NO:10 and the light chain consists of the amino acid sequence set forth in SEQ ID NO:11. In some embodiments, the anti-α-synuclein antibody is administered every 4 weeks, every 3 weeks, every 2 weeks, or every week. In some embodiments, 1 mg per kg of the anti-α-synuclein antibody is administered every 4 weeks or monthly. In some embodiments, 3 mg per kg of the anti-α-synuclein antibody is administered every 4 weeks or monthly. In some embodiments, 5 mg per kg of the anti-α-synuclein antibody is administered every 4 weeks or monthly. In some embodiments, 15 mg per kg of the anti-α-synuclein antibody is administered every 4 weeks or monthly. In some embodiments, 45 mg per kg of the anti-α-synuclein antibody is administered every 4 weeks or monthly. In some embodiments, 90 mg per kg of the anti-α-synuclein antibody is administered every 4 weeks or monthly. In some embodiments, 135 mg per kg of the anti-α-synuclein antibody is administered every 4 weeks or monthly. In certain embodiments, the human subject is administered at least 2 doses of the anti-α-synuclein antibody. In certain embodiments, the human subject is administered at least 4 doses of the anti-α-synuclein antibody. In certain embodiments, the human subject is administered at least 6 doses of the anti-α-synuclein antibody. In certain embodiments, the human subject is administered at least 8 doses of the anti-α-synuclein antibody. In certain embodiments, the human subject is administered at least 10 doses of the anti-α-synuclein antibody. In certain embodiments, the human subject is administered at least 12 doses of the anti-α-synuclein antibody. In certain embodiments of the above-described dosing regimens, the human subject is administered the anti-α-synuclein antibody for at least 1, 2, 3, 4, 5, 6, 7, 8, 9 ,10, 11, 12, 13, 14, 15, 16, 17, 18, 19, 20, or more years.

In a third aspect, the disclosure provides a sterile composition comprising a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg of an anti-α-synuclein antibody together with pharmaceutically acceptable carrier. The anti-α-synuclein antibody comprises an immunoglobulin heavy chain variable region (VH) and an immunoglobulin light chain variable region (VL), wherein: the VH comprises VH complementarity determining regions (VH-CDRs), wherein: VH-CDR1 consists of the amino acid residues of SEQ ID NO:1; VH-CDR2 consists of the amino acid residues of SEQ ID NO:2; and VH-CDR3 consists of the amino acid residues of SEQ ID NO:3; and the VL comprises VL-CDRs, wherein: VL-CDR1 consists of the amino acid residues of SEQ ID NO:4; VL-CDR2 consists of the amino acid residues of SEQ ID NO:5; and VL-CDR3 consists of the amino acid residues of SEQ ID NO:6. In some instances, the fixed doses are 250 mg, 1250 mg, and/or 3500 mg of the anti-α-synuclein antibody.

In certain embodiments of this aspect, the sterile composition is provided in a vial. In other embodiments, the sterile composition is provided in a syringe or pump adapted for intravenous administration of the anti-α-synuclein antibody. In certain embodiments, the VH consists of the amino acid sequence set forth in SEQ ID NO:8. In certain embodiments, the VL consists of the amino acid sequence set forth in SEQ ID NO:9. In certain embodiments, the VH consists of the amino acid sequence set forth in SEQ ID NO:8 and the VL consists of the amino acid sequence set forth in SEQ ID NO:9. In certain embodiments, the antibody comprises a human IgG1 heavy chain constant region. In some embodiments, the antibody comprises a human lambda light chain constant region. In certain embodiments, the antibody comprises a human IgG1 heavy chain constant region and a human lambda light chain constant region. In yet other embodiments, the antibody comprises a heavy chain and a light chain, wherein the heavy chain consists of the amino acid sequence set forth in SEQ ID NO:10 and the light chain consists of the amino acid sequence set forth in SEQ ID NO:11. In certain embodiments, the sterile composition comprises a fixed dose of 250 mg of the anti-α-synuclein antibody. In some embodiments, the sterile composition comprises a fixed dose of 1250 mg of the anti-α-synuclein antibody. In other embodiments, the sterile composition comprises a fixed dose of 3500 mg of the anti-α-synuclein antibody.

In a fourth aspect, featured is a method of treating a synucleinopathy in a human subject in need thereof The method comprises administering intravenously to the human subject the fixed dose of the anti-α-synuclein antibody from the sterile composition of the third aspect described above.

In certain embodiments of this aspect, the synucleinopathy is Parkinson's disease dementia (PDD), dementia with Lewy bodies (DLB), the Lewy body variant of Alzheimer's disease (LBVAD), multiple systems atrophy (MSA), pure autonomic failure (PAF), or neurodegeneration with brain iron accumulation type-1 (NBIA-I). In a particular embodiment, the synucleinopathy is Parkinson's disease. In certain cases, the PD is mild PD. In other cases, the PD is moderate PD. In some embodiments, the fixed dose is 250 mg of the anti-α-synuclein antibody. In some embodiments, the fixed dose is 1250 mg of the anti-α-synuclein antibody. In some embodiments, the fixed dose is 3500 mg of the anti-α-synuclein antibody.

In a fifth aspect, the disclosure relates to a method of treating abnormal accumulation or deposition of α-synuclein in the central nervous system in a human subject in need thereof. The method involves administering intravenously to the human subject the fixed dose of the anti-α-synuclein antibody from the sterile composition of the third aspect described above.

In certain embodiments, the human subject has been identified as having abnormal accumulation or deposition of α-synuclein in the central nervous system. In certain cases, the human subject is identified by in vivo imaging of α-synuclein (e.g., in the brain) by a method comprising positron emission tomography (PET), single photon emission tomography (SPECT), near infrared (NIR) optical imaging, magnetic resonance imaging (MRI), dopamine transporter imaging, or substantia nigra ultrasonography. In other cases, the human subject is identified by assaying the level of α-synuclein in a blood or plasma sample obtained from the subject following peripheral administration to the subject of the anti-α-synuclein antibody and comparing the assayed level of α-synuclein in the subject to a reference standard, wherein the difference or similarity between the level of α-synuclein in the blood or plasma sample and the reference standard correlates with the level of α-synuclein in the brain of the subject. In some embodiments, the fixed dose is 250 mg of the anti-α-synuclein antibody. In some embodiments, the fixed dose is 1250 mg of the anti-α-synuclein antibody. In some embodiments, the fixed dose is 3500 mg of the anti-α-synuclein antibody.

In some embodiments, the human subject is at risk of developing Parkinson's disease (e.g., due to the subject having a genetic risk factor such as a mutation in the SNCA, LRRK2, Parkin, PINK1, DJ1, ATP13A2, PLA2G6, FBXO7, UCHL1, GIGYF2, HTRA2, or EIF4G1 gene) or the human subject has prodromal Parkinson's disease (e.g., the subject has symptoms or clusters of symptoms associated with future development of Parkinson's disease such as hyposmia, REM Behavior Disorder, seborrheic dermatosis, and/or certain autonomic symptoms including but not limited to orthostatic hypotension, impotence in males, and/or disorders of bladder control).

These embodiments apply to the fourth and fifth aspects described above. In some embodiments, the anti-α-synuclein antibody is administered every 4 weeks, every 3 weeks, every 2 weeks, or every week. In some embodiments, a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg is administered every 4 weeks. In some embodiments, a fixed dose of 250 mg is administered every 4 weeks. In some embodiments, a fixed dose of 1250 mg is administered every 4 weeks. In some embodiments, a fixed dose of 3500 mg is administered every 4 weeks. In certain embodiments, the human subject is administered at least 2 doses of the anti-α-synuclein antibody. In certain embodiments, the human subject is administered at least 4 doses of the anti-α-synuclein antibody. In certain embodiments, the human subject is administered at least 6 doses of the anti-α-synuclein antibody. In certain embodiments, the human subject is administered at least 8 doses of the anti-α-synuclein antibody. In certain embodiments, the human subject is administered at least 10 doses of the anti-α-synuclein antibody. In certain embodiments, the human subject is administered at least 12 doses of the anti-α-synuclein antibody.

In a sixth aspect, the disclosure provides a method of treating a synucleinopathy in a human subject in need thereof. The method comprises administering intravenously to the human subject a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg of an anti-α-synuclein antibody. The anti-α-synuclein antibody comprises an immunoglobulin heavy chain variable region (VH) and an immunoglobulin light chain variable region (VL), wherein: the VH comprises VH complementarity determining regions (VH-CDRs), wherein: VH-CDR1 consists of the amino acid residues of SEQ ID NO:1; VH-CDR2 consists of the amino acid residues of SEQ ID NO:2; and VH-CDR3 consists of the amino acid residues of SEQ ID NO:3; and the VL comprises VL-CDRs, wherein: VL-CDR1 consists of the amino acid residues of SEQ ID NO:4; VL-CDR2 consists of the amino acid residues of SEQ ID NO:5; and VL-CDR3 consists of the amino acid residues of SEQ ID NO:6.

In certain embodiments, the synucleinopathy is Parkinson's disease dementia (PDD), dementia with Lewy bodies (DLB), the Lewy body variant of Alzheimer's disease (LBVAD), multiple systems atrophy (MSA), pure autonomic failure (PAF), or neurodegeneration with brain iron accumulation type-1 (NBIA-I). In a particular embodiment, the synucleinopathy is Parkinson's disease (PD). In some cases, the PD is mild PD. In other cases, the PD is moderate PD. In some embodiments, the method comprises administering intravenously to the human subject a fixed dose of 250 mg of the anti-α-synuclein antibody. In some embodiments, the method comprises administering intravenously to the human subject a fixed dose of 1250 mg of the anti-α-synuclein antibody. In some embodiments, the method comprises administering intravenously to the human subject a fixed dose of 3500 mg of the anti-α-synuclein antibody.

In a seventh aspect, the disclosure provides a method of treating abnormal accumulation or deposition of α-synuclein in the central nervous system in a human subject in need thereof The method involves administering intravenously to the human subject a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg of an anti-α-synuclein antibody. The anti-α-synuclein antibody comprises an immunoglobulin heavy chain variable region (VH) and an immunoglobulin light chain variable region (VL), wherein: the VH comprises VH complementarity determining regions (VH-CDRs), wherein: VH-CDR1 consists of the amino acid residues of SEQ ID NO:1; VH-CDR2 consists of the amino acid residues of SEQ ID NO:2; and VH-CDR3 consists of the amino acid residues of SEQ ID NO:3; and the VL comprises VL-CDRs, wherein: VL-CDR1 consists of the amino acid residues of SEQ ID NO:4; VL-CDR2 consists of the amino acid residues of SEQ ID NO:5; and VL-CDR3 consists of the amino acid residues of SEQ ID NO:6.

In some embodiments, the human subject is or has been identified as having abnormal accumulation or deposition of α-synuclein in the central nervous system. In certain cases, the human subject is identified by in vivo imaging of α-synuclein (e.g., in the brain) by a method comprising positron emission tomography (PET), single photon emission tomography (SPECT), near infrared (NIR) optical imaging, magnetic resonance imaging (MRI), dopamine transporter imaging, or substantia nigra ultrasonography. In other cases, the human subject is or has been identified by assaying the level of α-synuclein in a blood or plasma sample obtained from the subject following peripheral administration to the subject of the anti-α-synuclein antibody and comparing the assayed level of α-synuclein in the subject to a reference standard, wherein the difference or similarity between the level of α-synuclein in the blood or plasma sample and the reference standard correlates with the level of α-synuclein in the brain of the subject.

In some embodiments, the human subject is at risk of developing Parkinson's disease (e.g., due to the subject having a genetic risk factor such as a mutation in the SNCA, LRRK2, Parkin, PINK1, DJ1, ATP13A2, PLA2G6, FBXO7, UCHL1, GIGYF2, HTRA2, or EIF4G1 gene) or the human subject has prodromal Parkinson's disease (e.g., the subject has symptoms or clusters of symptoms associated with future development of Parkinson's disease such as hyposmia, REM Behavior Disorder, seborrheic dermatosis, and/or certain autonomic symptoms including but not limited to orthostatic hypotension, impotence in males, and/or disorders of bladder control). In some embodiments, the method comprises administering intravenously to the human subject a fixed dose of 250 mg of the anti-α-synuclein antibody. In some embodiments, the method comprises administering intravenously to the human subject a fixed dose of 1250 mg of the anti-α-synuclein antibody. In some embodiments, the method comprises administering intravenously to the human subject a fixed dose of 3500 mg of the anti-α-synuclein antibody.

These embodiments apply to both the sixth and seventh aspects described above. In certain embodiments, the VH consists of the amino acid sequence set forth in SEQ ID NO:8. In certain embodiments, the VL consists of the amino acid sequence set forth in SEQ ID NO:9. In certain embodiments, the VH consists of the amino acid sequence set forth in SEQ ID NO:8 and the VL consists of the amino acid sequence set forth in SEQ ID NO:9. In certain embodiments, the antibody comprises a human IgG1 heavy chain constant region. In some embodiments, the antibody comprises a human lambda light chain constant region. In certain embodiments, the antibody comprises a human IgG1 heavy chain constant region and a human lambda light chain constant region. In yet other embodiments, the antibody comprises a heavy chain and a light chain, wherein the heavy chain consists of the amino acid sequence set forth in SEQ ID NO:10 and the light chain consists of the amino acid sequence set forth in SEQ ID NO:11. In certain embodiments, the anti-α-synuclein antibody is administered monthly, every 4 weeks, every 3 weeks, every 2 weeks, or every week.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF DRAWINGS

FIG. 1 is a graph depicting the serum concentration (ng/ml) of BIIB054 in individual human subjects. Each curve in this graph corresponds to a different human subject.

FIG. 2 is a graph showing the mean serum profiles calculated for patients at each dose level at indicated times. The curve farthest from the x-axis corresponds to 135 mg/kg; the next to 90 mg/kg; the next to 45 mg/kg; the next to 15 mg/kg; the next to 5 mg/kg; and the curve closest to the x-axis to 1 mg/kg.

FIG. 3 is a graph showing the dose-dependency (in the dose range from 1 to 135 mg/kg) of the AUC.

FIG. 4 is a graph showing the dose-dependency (in the dose range from 1 to 135 mg/kg) of the Cmax.

FIG. 5 is a graph showing a dose response range for BIIB054 concentration in interstitial fluid (ISF) versus percent α-synuclein target binding.

FIG. 6 is a graph showing CSF concentrations vs. time for the 3, 15, and 45 mg/kg doses.

FIG. 7 is a graph showing simulated CSF concentration-time profiles for three doses.

### DETAILED DESCRIPTION

This disclosure features dosage regimens of anti-α-synuclein antibodies and α-synuclein-binding fragments thereof and their use in the treatment of synucleinopathies (e.g., disorders related to aggregates of α-synuclein such as Parkinson's disease (PD), Parkinson's Disease Dementia (PDD), dementia with Lewy bodies (DLB), Lewy body variant of Alzheimer's disease (AD), pure autonomic failure (PAF), multiple system atrophy (MSA), and neurodegeneration with brain iron accumulation type-1 (NBIA-I)).

### α-Synuclein

Synucleins are small, soluble proteins expressed primarily in neural tissue and in certain tumors. The family includes three known proteins: α-synuclein, β-synuclein, and γ-synuclein. All synucleins have in common a highly conserved α-helical lipid-binding motif with similarity to the class-A2 lipid-binding domains of the exchangeable apolipoproteins. Synuclein family members are not found outside vertebrates, although they have some conserved structural similarity with plant “late-embryo-abundant” proteins. The α- and β-synuclein proteins are found primarily in brain tissue, where they are seen mainly in presynaptic terminals. The γ-synuclein protein is found primarily in the peripheral nervous system and retina, but its expression in breast tumors is a marker for tumor progression. Normal cellular functions have not been determined for any of the synuclein proteins, although some data suggest a role in the regulation of membrane stability and/or turnover. Mutations in α-synuclein are associated with rare familial cases of early-onset Parkinson's disease, and the protein accumulates abnormally in Parkinson's disease, Alzheimer's disease, and several other neurodegenerative illnesses.

α-synuclein was originally identified in human brains as the precursor protein of the non-β-amyloid component of (NAC) of Alzheimer's disease (AD) plaques; see, e.g., Ueda et al, Proc. Natl. Acad. Sci. USA. 90 (1993), 1282-1286. α-synuclein, also termed the precursor of the non-Aβ component of AD amyloid (NACP), is a protein of 140 amino acids. α-synuclein exists in its native form as a random coil; however, changes in pH, molecular crowding, heavy metal content, and dopamine levels all affect protein conformation. Changes in conformation to oligomeric, proto-fibrillar, fibrillar, and aggregate moieties are thought to regulate protein toxicity. Increasing evidence indicates that dopamine-adducted α-synuclein has a faster time course to fibril formation compared to non-adducted protein. Furthermore, dopamine in the background of α-synuclein overexpression is toxic.

In this specification, the term “α-synuclein” is used to refer collectively to all types and forms of α-synuclein (e.g., the native monomer form of α-synuclein, other conformers of α-synuclein, for example, α-synuclein bonded to dopamine-quinone (DAQ), and oligomers or aggregates of α-synuclein).

The protein sequence for human α-synuclein is provided below: MDVFMKGLSKAKEGVVAAAEKTKQGVAEAAGKTKEGVLYVGSKTKEGVVHGVATVAEKTKEQ VTNVGGAVVTGVTAVAQKTVEGAGSIAAATGFVKKDQLGKNEEGAPQEGILEDMPVDPDNEA YEMPSEEGYQDYEPEA (SEQ ID NO:12). See, e.g., Ueda et al., ibid.; GenBank swissprot: locus SYUA_HUMAN, accession number P37840.

### Anti-α-Synuclein Antibodies

The anti-α-synuclein antibody or α-synuclein-binding fragment thereof used in the compositions and methods described herein bind α-synuclein, but not β-synuclein and/or γ-synuclein. Thus, although α-, β-, and γ-synuclein proteins are highly homologous proteins, the anti-α-synuclein antibody or α-synuclein-binding fragments described herein are specific for α-synuclein. These antibodies bind an N-terminal region of α-synuclein. Specifically, these antibodies bind an epitope within amino acids 4-15 of SEQ ID NO: 12 (i.e., FMKGLSKAKEGV (SEQ ID NO:13) and lysine at position 10 in SEQ ID NO:12 plays a significant role in the specificity of the antibodies disclosed herein for α-synuclein over (3-, and γ-synuclein proteins. In addition, the antibodies disclosed herein preferentially bind to pathological aggregates of human α-synuclein such as oligomers and fibrils of human α-synuclein over physiological human α-synuclein monomers. In certain cases, these antibodies can bind with high affinity to the A30P, E46K, and A53T mutant forms of human α-synuclein.

In some embodiments, the anti-α-synuclein antibody or α-synuclein-binding fragment thereof used in the compositions and methods described herein comprises the three heavy chain variable domain complementarity determining regions (CDRs) of an antibody referred to as BIIB054.

BIIB054 is an exemplary anti-α-synuclein antibody that can be used in the compositions and methods described herein. BIIB054 is a fully human IgG1/λ monoclonal antibody identified and cloned from B-lymphocytes obtained under informed consent from a cohort of healthy elderly subjects with an absence of clinical signs and symptoms associated with neurological or psychiatric disorders. BIIB054 binds with sub-nanomolar affinity to the N-terminal (amino acids 4-10 of SEQ ID NO:12: FMKGLSK (SEQ ID NO:14)) region of α-synuclein. The apparent binding affinity is higher for oligomeric/fibrillar species than for the monomeric species of α-synuclein due to multivalent binding. Importantly, BIIB054 does not bind to other highly homologous members of the synuclein family, e.g., β-synuclein, which can be neuroprotective. Immunohistochemistry shows specific (no off-target) binding of BIIB054 to Lewy bodies and Lewy neurites in both human Parkinson's patient and human α-synuclein transgenic mouse brain tissue. In transgenic mice overexpressing α-synuclein, intraperitoneal BIIB054 administration results in measurable drug levels in the brain as assessed by immunohistological and biochemical methods, showing BIIB054 crosses the blood-brain barrier.

In some embodiments, the anti-α-synuclein antibody or α-synuclein-binding fragment thereof comprises the three light chain variable domain CDRs of BIIB054. In some embodiments, the anti-α-synuclein antibody or α-synuclein-binding fragment thereof comprises the three heavy chain variable domain CDRs of BIIB054. In still other embodiments, the anti-α-synuclein antibody or α-synuclein-binding fragment thereof comprises the three heavy chain variable domain CDRs and the three light chain variable domain CDRs of BIIB054. The CDRs can be based on any CDR definition in the art, e.g., the definitions of Kabat, Chothia, Chothia from Abysis, enhanced Chothia/AbM, or based on the contact definition. CDR sequences of BIIB054 are provided in Table 1 below.

TABLE 1
Sequences cf the CDRs cf BIIB054

| Domain | Amino Acid Sequence |
|---|---|
| VH CDR1 | KAWMS(SEQ ID NO: 1) or |
| | GFDFEKAWMS (SEQ ID NO: 7) |
| VH CDR2 | RIKSTADGGTTSYAAPVEG (SEQ ID NO: 2) |
| VH CDR3 | AH (SEQ ID NO: 3) |
| VL CDR1 | SGEALPMQFAH (SEQ ID NO: 4) |
| VL CDR2 | KDSERPS (SEQ ID NO: 5) |
| VL CDR3 | QSPDSTNTYEV (SEQ ID NO: 6) |

In some embodiments, the anti-α-synuclein antibody or α-synuclein-binding fragment thereof comprises a VH CDR1 comprising or consisting of the amino acid sequence set forth in SEQ ID NO:1 or SEQ ID NO:7, a VH CDR2 comprising or consisting of the amino acid sequence set forth in SEQ ID NO:2; and a VH CDR3 comprising or consisting of the amino acid sequence set forth in SEQ ID NO:3. In some embodiments, the anti-α-synuclein antibody or α-synuclein-binding fragment thereof comprises a VL CDR1 comprising or consisting of the amino acid sequence set forth in SEQ ID NO:4, a VL CDR2 comprising or consisting of the amino acid sequence set forth in SEQ ID NO:5; and a VL CDR3 comprising or consisting of the amino acid sequence set forth in SEQ ID NO:6.

In some embodiments, the anti-α-synuclein antibody or α-synuclein-binding fragment thereof comprises a VH CDR1 comprising or consisting of the amino acid sequence set forth in SEQ ID NO:1 or SEQ ID NO:7, a VH CDR2 comprising or consisting of the amino acid sequence set forth in SEQ ID NO:2; and a VH CDR3 comprising or consisting of the amino acid sequence set forth in SEQ ID NO:3; a VL CDR1 comprising or consisting of the amino acid sequence set forth in SEQ ID NO:4, a VL CDR2 comprising or consisting of the amino acid sequence set forth in SEQ ID NO:5; and a VL CDR3 comprising or consisting of the amino acid sequence set forth in SEQ ID NO:6.

In some embodiments, the anti-α-synuclein antibody or α-synuclein-binding fragment thereof comprises or consists of the variable heavy chain (VH) of BIIB054. The VH of BIIB054 has the following amino acid sequence (VH-CDRs underlined):

| (SEQ ID NO: 8) | |
|---|---|
| 1 | EVQLVESGGG LVEPGGSLRL SCAVS [symbol] KAWMSWVRQA PGQGLQWVAR |
| 51 | IKSTADGGTT SYAAPVEGRF IISRDDSRNM LYLQMNSLKT EDTAVYYCTS |
| 101 | AHWGQGTLVT VSS |

In some embodiments, the anti-α-synuclein antibody or α-synuclein-binding fragment thereof comprises or consists of the variable light chain (VL) of BIIB054. The VL of BIIB054 has the following amino acid sequence (VL-CDRs underlined):

| (SEQ ID NO: 9) | |
|---|---|
| 1 | SYELTQPPSV SVSPGQTARI TCSGEALPMQ FAHWYQQRPG KAPVIVVYKD |
| 51 | SERPSGVPER FSGSSSGTTA TLTITGVQAE DEADYYCQSP DSTNTYEVFG |
| 101 | GGTKLTVL |

An antibody consisting of the mature heavy chain (SEQ ID NO:10) and the mature light chain (SEQ ID NO:11) listed below is termed “BIIB054” as used herein.

### Mature BIIB054 Heavy Chain (HC):

| (SEQ ID NO: 10) | |
|---|---|
| 1 | EVQLVESGGG LVEPGGSLRL SCAVS [symbol] KAWMSWVRQA PGQGLQWVAR |
| 51 | IKSTADGGTT SYAAPVEGRF IISRDDSRNM LYLQMNSLKT EDTAVYYCTS |
| 101 | AHWGQGTLVT VSSASTKGPS VFPLAPSSKS TSGGTAALGC LVKDYFPEPV |
| 151 | TVSWNSGALT SGVHTFPAVL QSSGLYSLSS VVTVPSSSLG TQTYICNVNH |
| 201 | KPSNTKVDKR VEPKSCDKTH TCPPCPAPEL LGGPSVFLFP PKPKDTLMIS |
| 251 | RTPEVTCVVV DVSHEDPEVK FNWYVDGVEV HNAKTKPREE QYNSTYRVVS |
| 301 | VLTVLHQDWL NGKEYKCKVS NKALPAPIEK TISKAKGQPR EPQVYTLPPS |
| 351 | REEMTKNQVS LTCLVKGFYP SDIAVEWESN GQPENNYKTT PPVLDSDGSF |
| 401 | FLYSKLTVDK SRWQQGNVFS CSVMHEALHN HYTQKSLSLS PG |

### Mature BIIB054 Light Chain (LC):

| (SEQ ID NO: 11) | |
|---|---|
| 1 | SYELTQPPSV SVSPGQTARI TCSGEALPMQ FAHWYQQRPG KAPVIVVYKD |
| 51 | SERPSGVPER FSGSSSGTTA TLTITGVQAE DEADYYCQSP DSTNTYEVFG |
| 101 | GGTKLTVLSQ PKAAPSVTLF PPSSEELQAN KATLVCLISD FYPGAVTVAW |
| 151 | KADSSPVKAG VETTTPSKQS NNKYAASSYL SLTPEQWKSH RSYSCQVTHE |
| 201 | GSTVEKTVAP TECS |

In the above-listed VH, VL, HC, and LC sequences, CDRs 1, 2, and 3 based on the Kabat definition are underlined. The italicized and boldened sequence in the VH and HC is the additional N-terminal sequence found in the CDR1 based on enhanced Chothia/AbM definition.

In certain embodiments of the methods and compositions disclosed herein, the anti-α-synuclein antibody or α-synuclein-binding fragment thereof comprises a VH having the amino acid sequence set forth in SEQ ID NO:8. In certain embodiments of the methods and compositions disclosed herein, the anti-α-synuclein antibody or α-synuclein-binding fragment thereof comprises a VL having the amino acid sequence set forth in SEQ ID NO:9. In certain embodiments of the methods and compositions disclosed herein, the anti-α-synuclein antibody or α-synuclein-binding fragment thereof comprises a VH having the amino acid sequence set forth in SEQ ID NO:8 and a VL having the amino acid sequence set forth in SEQ ID NO:9. In certain embodiments of the methods and compositions disclosed herein, the anti-α-synuclein antibody or α-synuclein-binding fragment thereof comprises a heavy chain having the amino acid sequence set forth in SEQ ID NO:10. In certain embodiments of the methods and compositions disclosed herein, the anti-α-synuclein antibody or α-synuclein-binding fragment thereof comprises a light chain having the amino acid sequence set forth in SEQ ID NO:11. In other embodiments of the methods and compositions disclosed herein, the anti-α-synuclein antibody or α-synuclein-binding fragment thereof comprises a heavy chain having the amino acid sequence set forth in SEQ ID NO:10 and a light chain having the amino acid sequence set forth in SEQ ID NO:11.

In some embodiments, the anti-α-synuclein antibody or α-synuclein-binding fragment thereof selectively binds to α-synuclein and comprises a HC that is at least 70%, 75%, 80%, 85%, 90%, 91%, 92%, 93%, 94%, 95%, 96%, 97%, 98%, 99% or more identical to the amino acid sequence of SEQ ID NO:10, or differs at least at 1 to 5 amino acid residues, but at fewer than 40, 30, 20, 15, or 10, residues, from SEQ ID NO:10. In one embodiment, the six CDRs are identical to the six CDRs of BIIB054 and any substitutions are made to the framework region.

In some embodiments, the anti-α-synuclein antibody or α-synuclein-binding fragment thereof selectively binds to α-synuclein and comprises a LC that is at least 70%, 75%, 80%, 85%, 90%, 91%, 92%, 93%, 94%, 95%, 96%, 97%, 98%, 99% or more identical to the amino acid sequence of SEQ ID NO:11, or differs at least at 1 to 5 amino acid residues, but at fewer than 40, 30, 20, 15, or 10, residues, from SEQ ID NO:11. In one embodiment, the six CDRs are identical to the six CDRs of BIIB054 and any substitutions are made to the framework region.

In certain embodiments, the anti-α-synuclein antibody is an IgG antibody. In specific embodiments, the anti-α-synuclein antibody has heavy chain constant region chosen from, e.g., IgG1, IgG2, IgG3, IgG4, IgM, IgAl, IgA2, IgD, and IgE. In one embodiment, the anti-α-synuclein antibody is of the IgG1 isotype. In another embodiment, the anti-α-synuclein antibody is of the IgG2 isotype. In yet another embodiment, the anti-α-synuclein antibody is of the IgG3 isotype. In further embodiments, the anti-α-synuclein antibody has a light chain constant region chosen from, e.g., a human kappa or human lambda light chain. In a certain embodiment, the anti-α-synuclein antibody is an IgG1/human lambda antibody.

In some embodiments, the anti-α-synuclein antibody is a full-length (whole) antibody or substantially full-length. The protein can include at least one, and preferably two, complete heavy chains, and at least one, and preferably two, complete light chains. In some embodiments, the anti-α-synuclein antibody is an α-synuclein-binding fragment. In some instances, the α-synuclein-binding fragment is a Fab, a Fab′, an F(ab′)2, a Facb, an Fv, a single chain Fv (scFv), a sc(Fv)2, or a diabody.

The heavy chain and light chain of the antibodies disclosed herein may also include signal sequences. The signal sequences can be selected from those known in the art, for example, MDMRVPAQLLGLLLLWFPGSRC (SEQ ID NO:15) or MDMRVPAQLLGLLLLWLPGARC (SEQ ID NO:16).

Antibodies, such as BIIB054, or α-synuclein-binding fragments thereof can be made, for example, by preparing and expressing synthetic genes that encode the recited amino acid sequences or by mutating human germline genes to provide a gene that encodes the recited amino acid sequences. Moreover, this antibody and other anti-α-synuclein antibodies can be produced, e.g., using one or more of the following methods.

### Methods of Producing Antibodies

Anti-α-synuclein antibodies or α-synuclein-binding fragments may be produced in bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. To produce the antibody of interest, a polynucleotide or polynucleotides encoding the antibody is/are constructed, introduced into an expression vector or expression vectors, and then expressed in suitable host cells. To improve expression, the nucleotide sequences of the light and heavy chain genes can be recoded without changing (or minimally changing—e.g., removal of a C-terminal residue of the heavy or light chain) the amino acid sequence. The areas for potential recoding include those associated with translation initiation, codon usage, and possible unintended mRNA splicing. Polynucleotides encoding an anti-α-synuclein antibody comprising the VH and/or VL, HC and/or LC of the α-synuclein antibodies described herein would be readily envisioned by the ordinarily skilled artisan.

An exemplary DNA sequence encoding the BIIB054 light chain is provided below (the nucleotides in lower case encode the native light chain signal peptide (which may or may not be included in the nucleic acid construct); the mature N-terminus begins with nucleic acid starting at position 67):

| (SEQ ID NO: 17) | |
|---|---|
| 1 | atg gac atg cgg gtg ccc gcc cag ctg ctg ggc ctg ctg ctg ctg tgg ctc cct ggc gcc aga tgt |
| 67 | AGC TAC GAG CTG ACC CAG CCC CCC AGC GTC AGC GTC AGC CCC GGC CAG ACC GCC AGG ATC ACC TGC |
| 133 | AGC GGC GAG GCC CTG CCC ATG CAG TTC GCC CAC TGG TAC CAA CAG AGG CCA GGC AAG GCC CCA GTG |
| 199 | ATC GTG GTG TAC AAA GAC AGT GAG AGA CCC TCA GGT GTC CCT GAG CGA TTC TCT GGC TCC TCT TCC |
| 265 | GGG ACA ACA GCC ACC TTG ACC ATC ACT GGA GTC CAG GCA GAA GAT GAG GCT GAC TAT TAC TGC CAG |
| 331 | TCT CCA GAC AGC ACT AAC ACT TAT GAA GTC TTC GGC GGA GGG ACC AAG CTG ACC GTC CTG AGT CAG |
| 397 | CCC AAG GCT GCC CCC TCC GTC ACT CTG TTC CCT CCC TCC TCC GAG GAA CTT CAA GCC AAC AAG GCC |
| 463 | ACA CTG GTC TGT CTC ATC AGT GAC TTC TAC CCT GGA GCC GTG ACA GTG GCC TGG AAG GCA GAT AGC |
| 529 | AGC CCC GTC AAG GCT GGA GTG GAG ACC ACC ACA CCC TCC AAA CAA AGC AAC AAC AAA TAC GCT GCC |
| 595 | AGC AGC TAC CTG AGC CTG ACA CCT GAG CAG TGG AAG TCC CAC AGA AGC TAC AGC TGC CAG GTC ACC |
| 661 | CAT GAA GGG AGC ACC GTG GAG AAG ACA GTG GCC CCT ACA GAA TGT TCA TAG |

An exemplary DNA sequence encoding the BIIB054 heavy chain is provided below (the nucleotides in lower case encode the native light chain signal peptide (which may or may not be included in the nucleic acid construct); the mature N-terminus begins with nucleic acid starting at position 67):

| (SEQ ID NO: 18) | |
|---|---|
| 1 | atg gac atg cgg gtg ccc gcc cag ctg ctg ggc ctg ctg ctg ctg tgg ttc ccc ggc tct cgg tgc |
| 67 | GAG GTG CAG CTG GTG GAG TCC GGG GGA GGT CTG GTC GAG CCT GGG GGG TCC CTG AGA CTC TCC TGT |
| 133 | GCA GTC TCC GGA TTC GAT TTC GAA AAA GCC TGG ATG AGT TGG GTC CGC CAG GCT CCA GGG CAG GGG |
| 199 | CTG CAG TGG GTT GCC CGG ATC AAG AGC ACA GCT GAT GGT GGG ACA ACA AGC TAC GCC GCC CCC GTG |
| 265 | GAA GGC AGA TTC ATC ATC TCA AGA GAT GAT TCC AGA AAC ATG CTT TAT CTG CAA ATG AAC AGT CTG |
| 331 | AAA ACT GAA GAC ACA GCC GTC TAT TAT TGT ACA TCA GCC CAC TGG GGC CAG GGA ACC CTG GTC ACC |
| 397 | GTC TCC TCT GCC TCC ACC AAG GGC CCA TCC GTC TTC CCT CTG GCA CCC TCC TCC AAA AGC ACC TCT |
| 463 | GGG GGC ACA GCC GCC CTG GGC TGC CTG GTC AAG GAC TAC TTC CCC GAA CCT GTG ACC GTC TCC TGG |
| 529 | AAC TCA GGC GCC CTG ACC AGC GGC GTG CAC ACC TTC CCT GCT GTC CTG CAA TCC TCC GGA CTC TAC |
| 595 | TCC CTC TCT TCC GTG GTG ACC GTG CCC TCC AGC AGC TTG GGC ACC CAG ACC TAC ATC TGC AAC GTG |
| 661 | AAT CAC AAG CCC AGC AAC ACC AAG GTG GAC AAG AGA GTT GAG CCC AAA TCT TGT GAC AAA ACT CAC |
| 727 | ACA TGC CCA CCC TGC CCA GCA CCT GAA CTC CTG GGG GGA CCC TCA GTC TTC CTC TTC CCC CCA AAA |
| 793 | CCC AAG GAC ACC CTC ATG ATC TCC CGG ACC CCT GAG GTC ACA TGC GTG GTG GTG GAC GTG AGC CAC |
| 859 | GAA GAC CCT GAG GTC AAG TTC AAC TGG TAT GTT GAC GGC GTG GAG GTC CAT AAT GCC AAG ACA AAG |
| 925 | CCT CGG GAG GAG CAG TAC AAC AGC ACC TAC CGC GTG GTC AGC GTC CTC ACC GTC CTG CAC CAA GAC |
| 991 | TGG CTG AAT GGC AAG GAG TAC AAG TGC AAG GTC TCC AAC AAA GCC CTC CCA GCC CCC ATC GAG AAA |
| 1057 | ACC ATC TCC AAA GCC AAA GGG CAG CCC CGA GAA CCA CAG GTG TAC ACC CTG CCC CCA TCC CGG GAG |
| 1123 | GAG ATG ACC AAG AAC CAA GTC AGC CTG ACC TGC CTG GTC AAA GGC TTC TAT CCC AGC GAC ATC GCC |
| 1189 | GTG GAG TGG GAG AGC AAT GGG CAG CCT GAG AAC AAC TAC AAG ACC ACA CCT CCC GTG CTG GAC TCC |
| 1255 | GAC GGC TCC TTC TTC CTC TAT TCC AAA CTC ACC GTG GAC AAG AGC AGG TGG CAG CAG GGG AAC GTC |
| 1321 | TTC TCA TGC TCC GTG ATG CAT GAG GCT CTG CAC AAC CAC TAC ACC CAG AAG AGC CTC TCC CTG TCC |
| 1387 | CCT GGT TGA |

Standard molecular biology techniques are used to prepare the recombinant expression vector(s), transfect the host cells, select for transformants, culture the host cells, and recover the antibody.

If the anti-α-synuclein antibodies or α-synuclein-binding fragments are to be expressed in bacterial cells (e.g., E. coli), the expression vector should have characteristics that permit amplification of the vector in the bacterial cells. Additionally, when E. coli such as JM109, DH5a, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)) (e.g., early simian virus 40 promoter), MMLV-LTR promoter, EF 1 a promoter (Mizushima et al., Nucleic Acids Res., 18:5322 (1990)), or CMV promoter (e.g., human cytomegalovirus immediate early promoter). In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell. In a specific embodiment, the mammalian cell is a CHO-DG441 cell.

In an exemplary system for antibody expression, a recombinant expression vector encoding both the antibody heavy chain and the antibody light chain of an anti-α-synuclein antibody (e.g., BIIB054) is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Dosing

The anti-α-synuclein antibody (e.g., BIIB054) can be administered to a subject, e.g., a human subject, at different doses. The anti-α-synuclein antibody (e.g., BIIB054) can be administered as a fixed dose (i.e., independent of the weight of the patient), or in a mg/kg dose (i.e., a dose which varies based on the weight of the subject). Dosage unit form or “fixed dose” as used herein refers to physically discrete units suited as unitary dosages for the subjects to be treated; each unit contains a predetermined quantity of antibody calculated to achieve the desired therapeutic concentration in the subject. Regardless of the form of the dose (e.g., per weight or fixed) the anti-α-synuclein antibody is administered in association with the required pharmaceutical carrier and optionally in association with another therapeutic agent. Single or multiple dosages may be given. The treatment can continue for days, weeks, months, a year, or even several years. The treatment can be part of a combination therapy in which anti-α-synuclein antibody is given in combination with one or more additional agents.

In one embodiment, for treating an indication described herein, the dosage of the anti-α-synuclein antibody is 1 mg/kg of body weight of the subject. In another embodiment, for treating an indication described herein, the dosage of the anti-α-synuclein antibody is 3 mg/kg of body weight of the subject. In yet another embodiment, for treating an indication described herein, the dosage of the anti-α-synuclein antibody is 5 mg/kg of body weight of the subject. In a further embodiment, for treating an indication described herein, the dosage of the anti-α-synuclein antibody is 15 mg/kg of body weight of the subject. In another embodiment, for treating an indication described herein, the dosage of the anti-α-synuclein antibody is 45 mg/kg of body weight of the subject. In yet another embodiment, for treating an indication described herein, the dosage of the anti-α-synuclein antibody is 90 mg/kg of body weight of the subject. In yet another embodiment, for treating an indication described herein, the dosage of the anti-α-synuclein antibody is 135 mg/kg of body weight of the subject. These doses may be prepared for administration in the form a sterile composition together with a pharmaceutically acceptable carrier and/or a beneficial excipient(s).

In one embodiment, for treating an indication described herein, the dosage of the anti-α-synuclein antibody is a fixed dose of 210 mg. In another embodiment, the dosage of the anti-α-synuclein antibody is a fixed dose of 225 mg. In another embodiment, the dosage of the anti-α-synuclein antibody is a fixed dose of 250 mg. In yet another embodiment, the dosage of the anti-α-synuclein antibody is a fixed dose of 350 mg. In another embodiment, the dosage of the anti-α-synuclein antibody is a fixed dose of 375 mg. In yet another embodiment, the dosage of the anti-α-synuclein antibody is a fixed dose of 1050 mg. In a further embodiment, the dosage of the anti-α-synuclein antibody is a fixed dose of 1125 mg. In another embodiment, the dosage of the anti-α-synuclein antibody is a fixed dose of 1250 mg. In another embodiment, of the dosage of the anti-α-synuclein antibody is a fixed dose of 3150 mg. In yet another embodiment, for treating an indication described herein, the dosage of the anti-α-synuclein antibody is a fixed dose of 3375 mg. In another embodiment, the dosage of the anti-α-synuclein antibody is a fixed dose of 3500 mg. In a further embodiment, the dosage of the anti-α-synuclein antibody is a fixed dose of 6300 mg. In another embodiment, the dosage of the anti-α-synuclein antibody is a fixed dose of 6750 mg. In another embodiment, the dosage of the anti-α-synuclein antibody is a fixed dose of 9450 mg. These fixed doses may be formulated in the form a sterile composition together with a pharmaceutically acceptable carrier and/or a beneficial excipient(s).

The mg/kg doses or fixed doses described above may each be administered to the subject daily, every week, every week-and-a-half, every 2 weeks, every two-and-a-half weeks, every 3 weeks, every 4 weeks, every 5 weeks, every 6 weeks, every 7 weeks, every 8 weeks, monthly, biweekly, weekly, or daily, as deemed appropriate by a health care provider, over a period of time to encompass at least 2 doses, 3 doses, 4 doses, 5 doses, 6 doses, 7 doses, 8 doses, 9 doses, 10 doses, 12 doses, 14 doses, 16 doses, 18 doses, 20 doses, 22 doses, 24 doses or more such that a desired therapeutic concentration is achieved and/or maintained in the subject.

The doses are administered intravenously.

Exemplary dosing regimens are provided in the table below:

| Route | Dose |
|---|---|
| IV, every 4 weeks (or monthly) | 3 mg/kg |
| IV, every 4 weeks (or monthly) | 15 mg/kg |
| IV, every 4 weeks (or monthly) | 45 mg/kg |

A pharmaceutical composition may include a “therapeutically effective amount” of agent described herein such that administration using a particular dosage regimen results in a therapeutically effective concentration of the antibody in the cerebrospinal fluid (CSF) or brain interstitial fluid (ISF). Such effective amounts can be determined based on the effect of the administered agent. A therapeutically effective amount of an agent may also vary according to factors such as the disease state, age, sex, and weight of the individual, and the ability of the compound to elicit a desired response in the individual. A therapeutically effective amount is also one in which any toxic, or detrimental effects, of the composition is outweighed by the therapeutically beneficial effects.

In one embodiment, intravenously administered anti-α-synuclein antibody (e.g., BIIB054) is dosed such that a desired therapeutic concentration of the antibody is achieved in CSF and/or ISF of the subject (e.g., human subject). In one embodiment, the antibody achieves a concentration sufficient to enter the brain and produce a therapeutic effect, e.g., mediated by, binding to aggregated α-synuclein and triggering microglia-dependent or -independent clearance/inactivation, reducing α-synuclein aggregation, and/or preventing prion-like intracellular spread of α-synuclein.

In another embodiment, the desired therapeutic concentration can be equal to the concentration of the anti-α-synuclein antibody (e.g., BIIB054) that is able to provide and maintain (after 1 or more doses) at least 30-50% reduction of aggregated α-synuclein in ISF and/or CSF of the subject.

In one embodiment, the desired therapeutic concentration achieves an anti-α-synuclein antibody (e.g., BIIB054) concentration which is at the EC₅₀ in the ISF of the subject.

In another embodiment, the desired therapeutic concentration can be equal to an anti-α-synuclein antibody (e.g., BIIB054) concentration that is able to provide and maintain (after 1 or more doses) 50-90% reduction of aggregated α-synuclein in ISF and/or CSF of the subject.

In one embodiment, the desired therapeutic concentration achieves an anti-α-synuclein antibody (e.g., BIIB054) concentration which is above the EC₅₀ and below the EC₉₀ in the ISF of the subject.

In still another embodiment, the desired therapeutic concentration can be equal to an anti-α-synuclein antibody (e.g., BIIB054) concentration that is able to provide and maintain (after 1 or more doses) greater than 90% reduction of aggregated α-synuclein in the ISF and/or CSF of the subject.

In one embodiment, the desired therapeutic concentration achieves an anti-α-synuclein antibody (e.g., BIIB054) concentration which is above the EC₉₀ in the ISF of the subject.

In one embodiment, a therapeutically effective dose is the dose of an anti-α-synuclein antibody (e.g., BIIB054) which can achieve and maintain the desired therapeutic concentration (after 1 or more doses).

### Methods of Treatment

The anti-α-synuclein antibodies described herein (e.g., BIIB054) can be used for the prophylactic and therapeutic treatment of a synucleinopathy in a subject (e.g., human subject) in need thereof.

Synucleinopathies include disorders related to aggregates of α-synuclein such as Parkinson's disease (PD), Parkinson's Disease Dementia (PDD), dementia with Lewy bodies (DLB), Lewy body variant of Alzheimer's disease (AD), pure autonomic failure (PAF), multiple system atrophy (MSA), and neurodegeneration with brain iron accumulation type-1 (NBIA-I)).

This disclosure also relates to a method of treating a neurological disorder characterized by abnormal accumulation and/or deposition of α-synuclein in the brain and the central nervous system, respectively, which method comprises administering to a subject (e.g., human subject) in need thereof a therapeutically effective amount of any one of the above-described α-synuclein antibodies. In certain embodiments, the neurological disorder is Parkinson's disease (PD), dementia with Lewy bodies (DLB), or multiple system atrophy (MSA).

Parkinson's disease is a clinical syndrome characterized by movement disorders and a range of non-motor features such as cognitive impairment. Pathologically there is marked cell loss in the locus ceruleus, dorsal motor nucleus of the vagus, raphe nucleus, substantia nigra pars compacta, nucleus basalis of Meynert and pedunculopontine nucleus, causing reductions in corresponding neurotransmitters. Cell loss is preceded by the formation of intracytoplasmic Lewy bodies (LB) and thickened neuritic processes referred to as Lewy neurites (LN). LNs can be found in areas without classical LBs including the amygdala, hippocampus and neocortex. There is no single cause of Parkinson's. Rather, there are multiple genetic and environmental causes. These causes collectively account for much of the risk and point to common, overlapping pathways of neurodegeneration. α-synuclein is a 14 kDa protein that is encoded in humans by the SNCA gene. Several lines of evidence point to a central role of α-synuclein in Parkinson's pathogenesis. First, neuropathological studies showed that aggregated α-synuclein is a core constituent of LBs and LNs. Genetic studies have identified three point mutations (A53T, A30P and E64K) that are associated with autosomal dominant Parkinson's. More recently triplications and duplications of SNCA were identified in patients with aggressive, early onset forms of Parkinson's. There is a link between seemingly sporadic Parkinson's and SNCA gene expression in that polymorphisms in the promoter region and reduced epigenetic silencing are associated with an increased risk of Parkinson's. Taken together, these genetic studies suggest a dose-response relationship and argue for a gain of α-synuclein function in Parkinson's. The toxicity of α-synuclein seems to be related to its propensity to aggregate. α-synuclein has a high propensity to aggregate in vitro. Autosomal dominant Parkinson's associated point mutations and multiplications increase the tendency for α-synuclein to polymerize and form oligomers and higher order fibrillar structures. Because of its central role in Parkinson's pathogenesis, approaches to modifying α-synuclein are an important potential target in Parkinson's and other synucleinopathies. Antibody-mediated removal and inactivation of α-synuclein can reduce aggregation and spreading of pathology and thereby slow the decline of clinical signs and symptoms of Parkinson's. A modest reduction of α-synuclein protein levels is enough to decrease Parkinson's progression. Indeed, α-synuclein gene duplication leads to an early onset familial Parkinson's but only to a 1.5-fold increase in α-synuclein proteins levels.

In one embodiment, provided is a method of treating a synucleinopathy, e.g., Parkinson's disease, in a human subject in need thereof. In certain embodiments, the Parkinson's disease is mild Parkinson's disease. In other embodiments, the Parkinson's disease is moderate Parkinson's disease. The method involves administering to the human subject a therapeutically effective amount of an anti-α-synuclein antibodies described herein (e.g., BIIB054). In certain instances, the subject is administered the anti-α-synuclein antibody at a dose of 3 mg per kg, 5 mg per kg, 15 mg per kg, 45 mg per kg, 90 mg per kg, or 135 mg per kg of body weight of the subject. In other instances, subject is administered the anti-α-synuclein antibody at a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg. In some instances, the subject is administered at least 2 doses, at least 3 doses, at least 4 doses, at least 5 doses, at least 6 doses, at least 7 doses, at least 8 doses, at least 9 doses, at least 10 doses, at least 11 doses, or at least 12 doses, or 2, 3, 4, 5, 6, 7, 8, 9, 10, 11, or 12 doses. Intravenously administered anti-α-synuclein antibody (e.g., BIIB054) can enter the brain, bind to α-synuclein, and trigger microglia-dependent or -independent clearance/inactivation, reduce α-synuclein aggregation, and/or prevent prion-like intracellular spread of α-synuclein. Peripheral anti-α-synuclein antibody (e.g., BIIB054) could also act as a peripheral sink for CNS α-synuclein.

In another embodiment, this disclosure features methods of treating a disorder characterized by aggregation and/or intracellular spread of α-synuclein. In certain embodiments, the disorder is multiple system atrophy (MSA). In other embodiments, the disorder is dementia with Lewy-bodies (DLB). The method involves administering to the human subject a therapeutically effective amount of an anti-α-synuclein antibodies described herein (e.g., BIIB054). In certain instances, the subject is administered the anti-α-synuclein antibody at a dose of 3 mg per kg, 5 mg per kg, 15 mg per kg, 45 mg per kg, 90 mg per kg, or 135 mg per kg of body weight of the subject. In other instances, subject is administered the anti-α-synuclein antibody at a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg. In some instances, the subject is administered at least 2 doses, at least 3 doses, at least 4 doses, at least 5 doses, at least 6 doses, at least 7 doses, at least 8 doses, at least 9 doses, at least 10 doses, at least 11 doses, or at least 12 doses, or 2, 3, 4, 5, 6, 7, 8, 9, 10, 11, or 12 doses.

In yet another embodiment, this disclosure relates to methods of treating a condition characterized by abnormal accumulation and/or deposition of α-synuclein in the central nervous system (e.g., brain) of a human subject in need thereof The method involves administering to the human subject a therapeutically effective amount of an anti-α-synuclein antibodies described herein (e.g., BIIB054). In certain instances, the subject is administered the anti-α-synuclein antibody at a dose of 3 mg per kg, 5 mg per kg, 15 mg per kg, 45 mg per kg, 90 mg per kg, or 135 mg per kg of body weight of the subject. In other instances, subject is administered the anti-α-synuclein antibody at a fixed dose of 210 mg, 225 mg, 250 mg, 350 mg, 375 mg, 1050 mg, 1125 mg, 1250 mg, 3150 mg, 3375 mg, 3500 mg, 6300 mg, 6750 mg, or 9450 mg. In some instances, the subject is administered at least 2 doses, at least 3 doses, at least 4 doses, at least 5 doses, at least 6 doses, at least 7 doses, at least 8 doses, at least 9 doses, at least 10 doses, at least 11 doses, or at least 12 doses, or 2, 3, 4, 5, 6, 7, 8, 9, 10, 11, or 12 doses.

In another embodiment, the disclosure features a method of treating a pre-symptomatic human subject in need of treatment to reduce or prevent abnormal accumulation and/or deposition of α-synuclein in the central nervous system (e.g., brain). The method involves administering to the human subject a therapeutically effective amount of an anti-α-synuclein antibodies described herein (e.g., BIIB054). In certain instances, the subject is administered the anti-α-synuclein antibody at a dose of 3 mg per kg, 5 mg per kg, 15 mg per kg, 45 mg per kg, 90 mg per kg, or 135 mg per kg of body weight of the subject. In other instances, subject is administered the anti-α-synuclein antibody at a fixed dose of 210 mg, 225 mg 250 mg, 350 mg, 375 mg, 1050 mg, 1125 mg, 1250 mg, 3150 mg, 3375 mg, 3500 mg, 6300 mg, 6750 mg, or 9450 mg. In some instances, the subject is administered at least 2 doses, at least 3 doses, at least 4 doses, at least 5 doses, at least 6 doses, at least 7 doses, at least 8 doses, at least 9 doses, at least 10 doses, at least 11 doses, or at least 12 doses, or 2, 3, 4, 5, 6, 7, 8, 9, 10, 11, or 12 doses.

A human subject can be identified as having abnormal accumulation or deposition of α-synuclein in the central nervous system (e.g., in the brain) by any method known in the art. In certain embodiments, the level of α-synuclein is assessed by in vivo imaging of α-synuclein (e.g., in the brain) and comprises positron emission tomography (PET), single photon emission tomography (SPECT), near infrared (NIR) optical imaging, magnetic resonance imaging (MRI), dopamine transporter imaging, or substantia nigra ultrasonography. In some of these embodiments, a labeled anti-α-synuclein antibody (e.g., labeled BIIB054) or an α-synuclein-binding fragment thereof is administered to a human subject and binding of the antibody to α-synuclein is assessed. The level of α-synuclein may also be assessed by other methods known in the art comprising, e.g., analyzing α-synuclein by one or more techniques chosen from Western blot, immunoprecipitation, enzyme-linked immunosorbent assay (ELISA), radioimmunoassay (RIA), fluorescent activated cell sorting (FACS), two-dimensional gel electrophoresis, mass spectroscopy (MS), matrix-assisted laser desorption/ionization-time of flight-MS (MALDI-TOF), surface-enhanced laser desorption ionization-time of flight (SELDI-TOF), high performance liquid chromatography (HPLC), fast protein liquid chromatography (FPLC), multidimensional liquid chromatography (LC) followed by tandem mass spectrometry (MS/MS), and laser densitometry. In certain instances, the level of α-synuclein in the brain of a subject can be assessed by assaying the level of α-synuclein in a blood or plasma sample obtained from the subject following peripheral administration to the subject of an anti-α-synuclein antibody (e.g., BIIB054) or an α-synuclein-binding fragment thereof and comparing the assayed level of α-synuclein in the subject to a reference standard, wherein the difference or similarity between the level of α-synuclein in the blood or plasma sample and the reference standard correlates with the level of α-synuclein in the brain of the subject.

In some embodiments in all of the above-described methods of treatment, the human subject is at risk of developing Parkinson's disease (e.g., due to the subject having a genetic risk factor such as a mutation in the SNCA, LRRK2, Parkin, PINK1, DJ1, ATP13A2, PLA2G6, FBXO7, UCHL1, GIGYF2, HTRA2, or EIF4G1 gene) or the human subject has prodromal Parkinson's disease (e.g., the subject has symptoms or clusters of symptoms associated with future development of Parkinson's disease such as hyposmia, REM Behavior Disorder, seborrheic dermatosis, and/or certain autonomic symptoms including but not limited to orthostatic hypotension, impotence in males, and/or disorders of bladder control).

In some embodiments in all of the above-described methods of treatment, the anti-α-synuclein antibody or antigen-binding fragment thereof selectively binds to α-synuclein and comprises (i) a VH domain that is at least 70%, 75%, 80%, 85%, 90%, 91%, 92%, 93%, 94%, 95%, 96%, 97%, 98%, 99% or more identical to the amino acid sequence of the VH domain of BIIB054 (SEQ ID NO:8), and/or (ii) a VL domain that is at least 70%, 75%, 80%, 85%, 90%, 91%, 92%, 93%, 94%, 95%, 96%, 97%, 98%, 99% or more identical to the amino acid sequence of the VL domain of BIIB054 (SEQ ID NO:9); or differs at least at 1 to 5 amino acid residues, but at fewer than 40, 30, 20, 15, or 10, residues, from SEQ ID NO:8 and/or SEQ ID NO:9. In certain embodiments, the anti-α-synuclein antibody or antigen-binding fragment thereof has six CDRs that are identical to the six CDRs of BIIB054 and any substitutions are made to the framework region. In certain instances, these anti-α-synuclein antibodies or α-synuclein-binding fragments (i) bind α-synuclein but do not significantly bind β-, or γ-synuclein; and/or (ii) selectively binds to an epitope within amino acids 4-15 of SEQ ID NO:12. In certain embodiments, the anti-α-synuclein antibody or antigen-binding fragment thereof comprises a VH domain consisting of the amino acid sequence set forth in SEQ ID NO:8 and a VL domain consisting of the amino acid sequence set forth in SEQ ID NO:9.

In certain embodiments in all of the above-described methods of treatment, the anti-α-synuclein antibody or α-synuclein-binding fragment thereof selectively binds to human α-synuclein and comprises (i) a heavy chain that is at least 70%, 75%, 80%, 85%, 90%, 91%, 92%, 93%, 94%, 95%, 96%, 97%, 98%, 99% or more identical to the amino acid sequence of SEQ ID NO:10, and/or (ii) a light chain that is at least 70%, 75%, 80%, 85%, 90%, 91%, 92%, 93%, 94%, 95%, 96%, 97%, 98%, 99% or more identical to the amino acid sequence of SEQ ID NO:11, or differs at least at 1 to 5 amino acid residues, but at fewer than 40, 30, 20, 15, or 10, residues, from SEQ ID NO:10 and/or SEQ ID NO:11. In certain embodiments, the anti-α-synuclein antibody comprises a heavy chain consisting of the amino acid sequence set forth in SEQ ID NO:10 and a light chain consisting of the amino acid sequence set forth in SEQ ID NO:11.

The following example is not to be construed as limiting the scope of the invention in any way.

### EXAMPLES

### Example 1

### Rationale for Dosing Regimen for BIIB054

Dosing regimens were selected based on safety, tolerability, pharmacokinetics (PK) data in serum and the cerebrospinal fluid (CSF), BIIB054 affinity to aggregated α-synuclein, and simulated BIIB054 CSF concentrations after multiple doses.

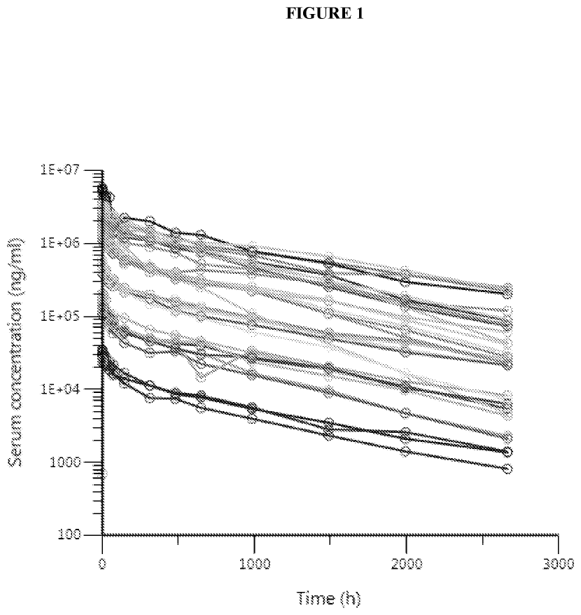

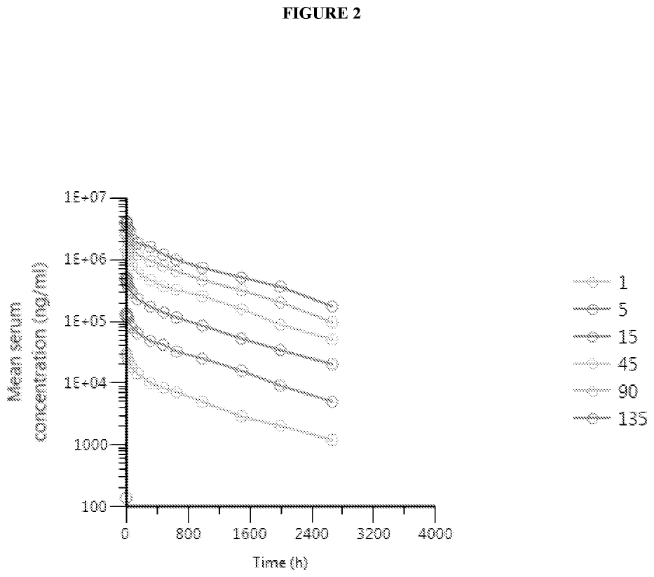

Single intravenous (IV) doses of BIIB054 up to and including 90 mg/kg in healthy subjects demonstrated acceptable tolerability. Serum profiles for BIIB054 were measured in subjects. A graph showing the serum concentration (ng/ml) in individual subjects are shown in FIG. 1. The mean serum profiles were calculated for patients at each dose level at indicated times and is depicted in FIG. 2.

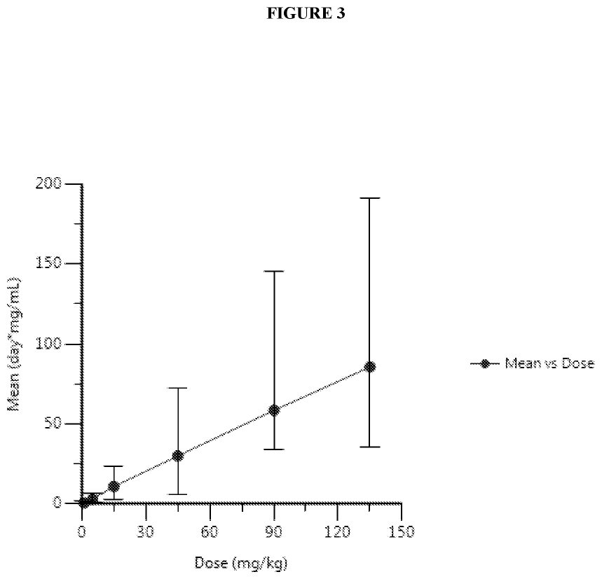

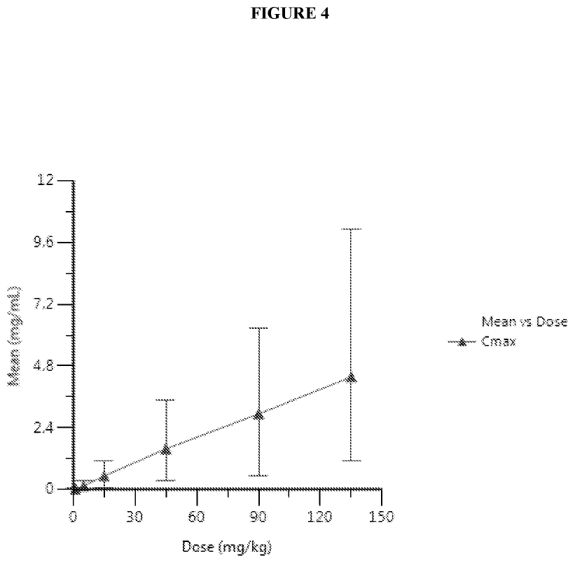

PK parameters as measured by AUC and Cmax were found to change in a dose-dependent manner in the dose range from 1 mg/kg to 135 mg/kg. The dose-dependency of the AUC is shown in FIG. 3 and of Cmax is shown in FIG. 4.

BIIB054 CSF concentrations after multiple doses were simulated based on a population PK model developed based on data from single IV doses.

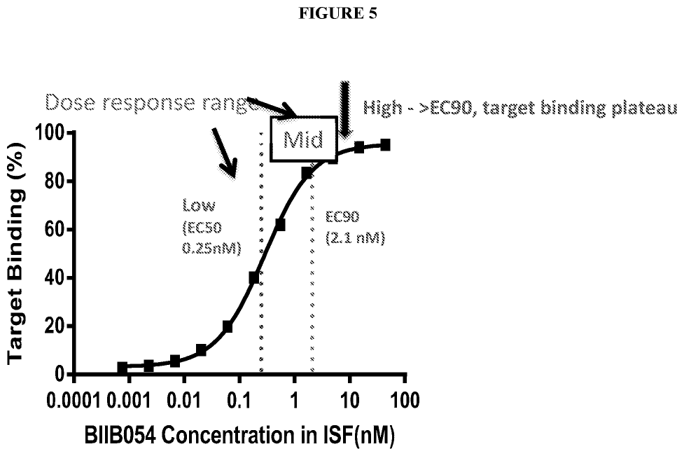

EC₅₀ and EC₉₀ values for BIIB054 to aggregated α-synuclein were derived from in vitro binding constants of the antibody to aggregated α-synuclein. Using these values, a dose response range for BIIB054 concentration in interstitial fluid (ISF) versus percent α-synuclein target binding was plotted and is shown in FIG. 5.

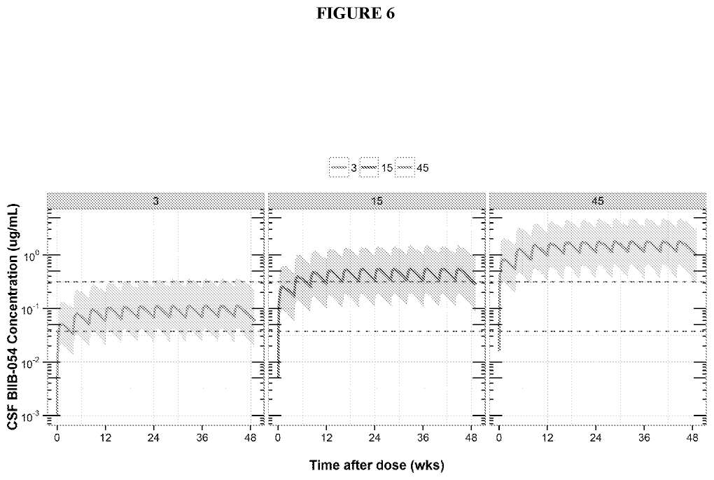

BIIB054 doses of 3, 15, and 45 mg/kg delivered as IV infusion every 4 weeks are supported by the following modeling using the single ascending dose (SAD) data. FIG. 6 shows CSF concentrations vs. time for the 3, 15, and 45 mg/kg doses. Based on the modeling:

(i) 3 mg/kg IV infusion once every 4 weeks was selected to maintain BIIB054 concentration in CSF and brain interstitial fluid (ISF) at or above EC₅₀ for the majority of subjects.

(ii) 15 mg/kg IV infusion once every 4 weeks was selected to maintain a CSF and ISF target at or above EC₉₀, as well as provide adequate separation between doses to elucidate the exposure-response relationship.

(iii) 45 mg/kg IV infusion once every 4 weeks was selected to maintain a CSF and ISF target above EC₉₀ for all subjects.

### Example 2

### Dose Selection for Phase 2

In this Phase 2 study, patients will receive an intravenous (IV) infusion of a study treatment (250, 1250, or 3500 mg) once every 4 weeks, for a total of 13 doses. The dose levels of BIIB054 were selected based on the safety, tolerability, and pharmacokinetics (PK) of BIIB054 in healthy volunteers, nonclinical toxicology data, BIIB054 affinity to aggregated alpha-synuclein in vitro, and simulated BIIB054 CSF concentration-time profiles in steady-state.

In vitro studies established that BIIB054 binds to both soluble and aggregated forms of α-synuclein, with a higher apparent binding affinity for aggregates. The half-maximal effective concentration (EC₅₀) of BIIB054 for aggregated α-synuclein was estimated at ˜0.25 nM, EC₉₀ was ˜2.1 nM (0.0375 μg/ml and 0.315 μg/ml, respectively).

A Phase 1, first-in-human study to evaluate single-ascending doses in healthy volunteers (HVs), ages 40 to 65 years, and subjects with PD is ongoing. HVs received IV doses from 1 mg/kg to 135 mg/kg, or placebo. Serum and CSF concentrations in HVs were described using a population PK model. Subsequently, estimated PK parameters as well as between subject variability and residual variability estimates from HVs were used to simulate 1000 serum and CSF steady-state profiles. To account for weight differences between HV and the target Phase 2 population, PPMI database was used as a source of weigh distribution in PD patients.

Simulations of CSF profiles were conducted for several dose levels between 1 mg/kg and 45 mg/kg to enable dose selection. CSF and brain interstitial fluid (ISF) concentrations of BIIB054 were assumed to be equal. Given the favorable safety profile of BIIB054, a fixed dose approach is to be implemented in this study.

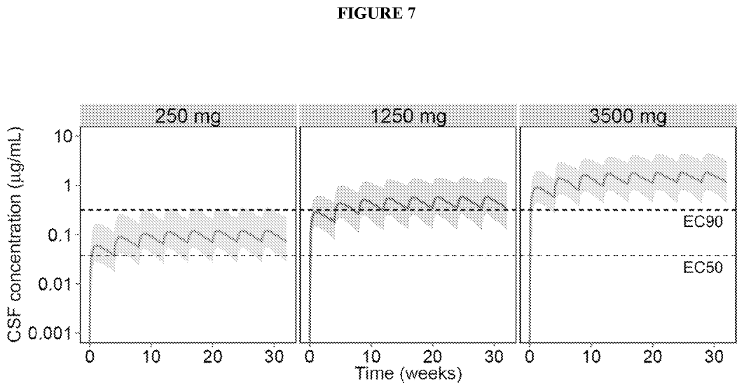

Simulations based on preliminary serum and CSF data from the first-in-human study indicate that for a 250 mg dose, BIIB054 concentration in CSF and ISF at steady state is projected to be above EC₅₀, for the majority of subjects. The highest dose (3500 mg) was selected to maintain these levels at above EC₉₀ for the 95% of subjects to increase the likelihood of demonstrating efficacy for BIIB054. An intermediate dose of 1250 mg is expected to maintain a CSF and ISF level at or above EC₉₀, as well as provide adequate separation between doses to elucidate the exposure-response relationship. See, FIG. 7. Summary statistics of simulated steady-state trough CSF concentrations for proposed Phase 2 doses are shown in Table 2.

TABLE 2
Summary statistics of simulated steady-state trough CSF
concentrations (μg/ml) for proposed Phase 2 doses.

| | Simulated steady-state trough CSF concentrations (μg/ml) | | |
|---|---|---|---|
| Dose, mg | Median, q50 | q5 | q95 |
| 250 | 0.076 | 0.029 | 0.191 |
| 1250 | 0.369 | 0.128 | 0.936 |
| 3500 | 1.07 | 0.403 | 2.79 |

Nonclinical efficacy data also suggest that the dose of 250 mg is expected to provide minimal efficacy based on studies in D-Line synuclein transgenic mouse. The estimated efficacious exposure in mouse was approximately 1317 day*m/mL. Clearance of BIIB054 in HV is on average 0.0052 L/h or 0.1248 L/day. Thus, using Dose=Cl×AUC, the projected mean minimum pharmacologically efficacious dose is approximately 164 mg.

Overall, all three doses are expected to be safe and well tolerated in humans. The highest planned dose (3500 mg) is expected to yield mean steady state area under the concentration-time curve from time zero to the time of next dosing (AUCₜₐₙ) and maximum observed concentration (Cₘₐₓ) values approximately 2.3- to 11-fold lower than those observed at the no observed adverse effect level in the 26-week toxicology study in rats (Table 3).

TABLE 3
Projected steady-state serum AUC_{tau,} steady-state
Cmax and safety margins for proposed Phase 2 doses.

| | Projected parameters | | Safety margins* | |
|---|---|---|---|---|
| | AUC_{tau,} h*μg/ml | Cmax, μg/ml | AUCₜₐᵤ | Cmax |
| | Median | Median | Median | Median |
| Dose, mg | (q5-q95) | (q5-q95) | (q5-q95) | (q5-q95) |
| 250 | 44400 | 134.4 | 31 | 149 |
| | (28000-68300) | (91.5-199.3) | (20-50) | (100-219) |
| 1250 | 220000 | 668 | 6.3 | 59 |
| | (134000-334000) | (448-1020) | (4.2-10) | (20-45) |
| 3500 | 610000 | 1860 | 2.3 | 11 |
| | (383000-937000) | (1230-2770) | (1.5-3.6) | (7-16) |

*Calculated based on mean AUC_{0-168 h} and Cₘₐₓ after the last dose of BIIB054 in 26-week rat toxicology study. AUCₜₐᵤ at NOAEL = AUC_{0-168 h}*4 = 1,395,000 h*μg/ml

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a synucleinopathy in a human subject in need thereof, the method comprising administering intravenously to the human subject an anti-α-synuclein antibody at a dose of 3 mg per kg, 5 mg per kg, 15 mg per kg, 45 mg per kg, or 90 mg per kg of body weight of the human subject, wherein the anti-α-synuclein antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid residues of SEQ ID NO:1;
VH-CDR2 consists of the amino acid residues of SEQ ID NO:2; and
VH-CDR3 consists of the amino acid residues of SEQ ID NO:3; and
(b) the VL comprises VL-CDRs, wherein:
VL-CDR1 consists of the amino acid residues of SEQ ID NO:4;
VL-CDR2 consists of the amino acid residues of SEQ ID NO:5; and
VL-CDR3 consists of the amino acid residues of SEQ ID NO:6.

2. The method of claim 1, wherein the synucleinopathy is Parkinson's disease (PD), Parkinson's disease dementia (PDD), dementia with Lewy bodies (DLB), the Lewy body variant of Alzheimer's disease (LBVAD), multiple systems atrophy (MSA), pure autonomic failure (PAF), or neurodegeneration with brain iron accumulation type-1 (NBIA-I).

3. The method of claim 1, wherein the synucleinopathy is Parkinson's disease.

4. A method of treating abnormal accumulation or deposition of α-synuclein in the central nervous system in a human subject in need thereof, the method comprising administering intravenously to the human subject an anti-α-synuclein antibody at a dose of 3 mg per kg, 5 mg per kg, 15 mg per kg, 45 mg per kg, or 90 mg per kg of body weight of the human subject, wherein the anti-α-synuclein antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid residues of SEQ ID NO:1;
VH-CDR2 consists of the amino acid residues of SEQ ID NO:2; and
VH-CDR3 consists of the amino acid residues of SEQ ID NO:3; and
(b) the VL comprises VL-CDRs, wherein:
VL-CDR1 consists of the amino acid residues of SEQ ID NO:4;
VL-CDR2 consists of the amino acid residues of SEQ ID NO:5; and
VL-CDR3 consists of the amino acid residues of SEQ ID NO:6.

5. The method of claim 4, wherein the human subject has been identified as having abnormal accumulation or deposition of α-synuclein in the central nervous system.

6. The method of any one of claims 1 to 5, wherein the VH consists of the amino acid sequence set forth in SEQ ID NO:8.

7. The method of any one of claims 1 to 5, wherein the VL consists of the amino acid sequence set forth in SEQ ID NO:9.

8. The method of any one of claims 1 to 5, wherein the VH consists of the amino acid sequence set forth in SEQ ID NO:8 and the VL consists of the amino acid sequence set forth in SEQ ID NO:9.

9. The method of any one of claims 1 to 8, wherein the antibody comprises a human IgG1 heavy chain constant region.

10. The method of any one of claims 1 to 8, wherein the antibody comprises a human lambda light chain constant region.

11. The method of any one of claims 1 to 5, wherein the antibody comprises a heavy chain and a light chain, wherein the heavy chain consists of the amino acid sequence set forth in SEQ ID NO:10 and the light chain consists of the amino acid sequence set forth in SEQ ID NO:11.

12. The method of any one of claims 1 to 11, wherein the anti-α-synuclein antibody is administered every 4 weeks, every 3 weeks, every 2 weeks, or every week.

13. The method of any one of claims 1 to 11, wherein 3 mg per kg of the anti-α-synuclein antibody is administered every 4 weeks.

14. The method of any one of claims 1 to 11, wherein 5 mg per kg of the anti-α-synuclein antibody is administered every 4 weeks.

15. The method of any one of claims 1 to 11, wherein 15 mg per kg of the anti-α-synuclein antibody is administered every 4 weeks.

16. The method of any one of claims 1 to 11, wherein 45 mg per kg of the anti-α-synuclein antibody is administered every 4 weeks.

17. The method of any one of claims 1 to 11, wherein 90 mg per kg of the anti-α-synuclein antibody is administered every 4 weeks.

18. The method of any one of claims 1 to 17, wherein the human subject is administered at least 2 doses of the anti-α-synuclein antibody.

19. The method of any one of claims 1 to 17, wherein the human subject is administered at least 12 doses of the anti-α-synuclein antibody.

20. A sterile composition comprising a fixed dose of 210 mg, 225 mg, 250 mg, 350 mg, 375 mg, 1050 mg, 1125 mg, 1250 mg, 3150 mg, 3375 mg, 3500 mg, 6300 mg, or 6750 mg of an anti-α-synuclein antibody together with pharmaceutically acceptable carrier, wherein the anti-α-synuclein antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid residues of SEQ ID NO:1;
VH-CDR2 consists of the amino acid residues of SEQ ID NO:2; and
VH-CDR3 consists of the amino acid residues of SEQ ID NO:3; and
(b) the VL comprises VL-CDRs, wherein:
VL-CDR1 consists of the amino acid residues of SEQ ID NO:4;
VL-CDR2 consists of the amino acid residues of SEQ ID NO:5; and
VL-CDR3 consists of the amino acid residues of SEQ ID NO:6.

21. The sterile composition of claim 20, wherein the sterile composition is provided in a vial.

22. The sterile composition of claim 20, wherein the sterile composition is provided in a syringe or pump adapted for intravenous administration of the anti-α-synuclein antibody.

23. The sterile composition of any one of claims 20 to 22, wherein the VH consists of the amino acid sequence set forth in SEQ ID NO:8.

24. The sterile composition of any one of claims 20 to 22, wherein the VL consists of the amino acid sequence set forth in SEQ ID NO:9.

25. The sterile composition of any one of claims 20 to 22, wherein the VH consists of the amino acid sequence set forth in SEQ ID NO:8 and the VL consists of the amino acid sequence set forth in SEQ ID NO:9.

26. The sterile composition of any one of claims 20 to 25, wherein the antibody comprises a human IgG1 heavy chain constant region.

27. The sterile composition of any one of claims 20 to 25, wherein the antibody comprises a human lambda light chain constant region.

28. The sterile composition of any one of claims 20 to 22, wherein the antibody comprises a heavy chain and a light chain, wherein the heavy chain consists of the amino acid sequence set forth in SEQ ID NO:10 and the light chain consists of the amino acid sequence set forth in SEQ ID NO:11.

29. A method of treating a synucleinopathy in a human subject in need thereof, the method comprising administering intravenously to the human subject the fixed dose of the anti-α-synuclein antibody from the sterile composition of any one of claims 20 to 28.

30. The method of claim 29, wherein the synucleinopathy is Parkinson's disease (PD), Parkinson's disease dementia (PDD), dementia with Lewy bodies (DLB), the Lewy body variant of Alzheimer's disease (LBVAD), multiple systems atrophy (MSA), pure autonomic failure (PAF), or neurodegeneration with brain iron accumulation type-1 (NBIA-I).

31. The method of claim 29, wherein the synucleinopathy is Parkinson's disease.

32. A method of treating abnormal accumulation or deposition of α-synuclein in the central nervous system in a human subject in need thereof, the method comprising administering intravenously to the human subject the fixed dose of the anti-α-synuclein antibody from the sterile composition of any one of claims 20 to 28.

33. The method of claim 32, wherein the human subject has been identified as having abnormal accumulation or deposition of α-synuclein in the central nervous system.

34. The method of any one of claims 29 to 33, wherein the anti-α-synuclein antibody is administered every 4 weeks, every 3 weeks, every 2 weeks, or every week.

35. The method of any one of claims 29 to 33, wherein a fixed dose of 210 mg, 225 mg, 250 mg, 350 mg, 375 mg, 1050 mg, 1125 mg, 1250 mg, 3150 mg, 3375 mg, 3500 mg, 6300 mg, or 6750 mg is administered every 4 weeks.

36. The method of any one of claims 29 to 35, wherein the human subject is administered at least 2 doses of the anti-α-synuclein antibody.

37. The method of any one of claims 29 to 35, wherein the human subject is administered at least 12 doses of the anti-α-synuclein antibody.

38. A method of treating a synucleinopathy in a human subject in need thereof, the method comprising administering intravenously to the human subject a fixed dose of 210 mg, 225 mg, 250 mg, 350 mg, 375 mg, 1050 mg, 1125 mg, 1250 mg, 3150 mg, 3375 mg, 3500 mg, 6300 mg, or 6750 mg of an anti-α-synuclein antibody, wherein the anti-α-synuclein antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid residues of SEQ ID NO:1;
VH-CDR2 consists of the amino acid residues of SEQ ID NO:2; and
VH-CDR3 consists of the amino acid residues of SEQ ID NO:3; and
(b) the VL comprises VL-CDRs, wherein:
VL-CDR1 consists of the amino acid residues of SEQ ID NO:4;
VL-CDR2 consists of the amino acid residues of SEQ ID NO:5; and
VL-CDR3 consists of the amino acid residues of SEQ ID NO:6.

39. The method of claim 38, wherein the synucleinopathy is Parkinson's disease (PD), Parkinson's disease dementia (PDD), dementia with Lewy bodies (DLB), the Lewy body variant of Alzheimer's disease (LBVAD), multiple systems atrophy (MSA), pure autonomic failure (PAF), or neurodegeneration with brain iron accumulation type-1 (NBIA-I).

40. The method of claim 38, wherein the synucleinopathy is Parkinson's disease.

41. A method of treating abnormal accumulation or deposition of α-synuclein in the central nervous system in a human subject in need thereof, the method comprising administering intravenously to the human subject a fixed dose of 210 mg, 225 mg, 250 mg, 350 mg, 375 mg, 1050 mg, 1125 mg, 1250 mg, 3150 mg, 3375 mg, 3500 mg, 6300 mg, or 6750 mg of an anti-α-synuclein antibody, wherein the anti-α-synuclein antibody comprises an immunoglobulin heavy chain variable region (VH) and an immunoglobulin light chain variable region (VL), wherein:
(a) the VH comprises VH complementarity determining regions (VH-CDRs), wherein:
VH-CDR1 consists of the amino acid residues of SEQ ID NO:1;
VH-CDR2 consists of the amino acid residues of SEQ ID NO:2; and
VH-CDR3 consists of the amino acid residues of SEQ ID NO:3; and
(b) the VL comprises VL-CDRs, wherein:
VL-CDR1 consists of the amino acid residues of SEQ ID NO:4;
VL-CDR2 consists of the amino acid residues of SEQ ID NO:5; and
VL-CDR3 consists of the amino acid residues of SEQ ID NO:6.

42. The method of claim 41, wherein the human subject has been identified as having abnormal accumulation or deposition of α-synuclein in the central nervous system.

43. The method of any one of claims 38 to 42, wherein the VH consists of the amino acid sequence set forth in SEQ ID NO:8.

44. The method of any one of claims 38 to 42, wherein the VL consists of the amino acid sequence set forth in SEQ ID NO:9.

45. The method of any one of claims 38 to 42, wherein the VH consists of the amino acid sequence set forth in SEQ ID NO:8 and the VL consists of the amino acid sequence set forth in SEQ ID NO:9.

46. The method of any one of claims 38 to 45, wherein the antibody comprises a human IgG1 heavy chain constant region.

47. The method of any one of claims 38 to 45, wherein the antibody comprises a human lambda light chain constant region.

48. The method of any one of claims 38 to 42, wherein the antibody comprises a heavy chain and a light chain, wherein the heavy chain consists of the amino acid sequence set forth in SEQ ID NO:10 and the light chain consists of the amino acid sequence set forth in SEQ ID NO:11.

49. The method of any one of claims 38 to 48, wherein the anti-α-synuclein antibody is administered every 4 weeks, every 3 weeks, every 2 weeks, or every week.

50. The sterile composition of any one of claims 20 to 28, comprising a fixed dose of 250 mg of the anti-α-synuclein antibody.

51. The sterile composition of any one of claims 20 to 28, comprising a fixed dose of 1250 mg of the anti-α-synuclein antibody.

52. The sterile composition of any one of claims 20 to 28, comprising a fixed dose of 3500 mg of the anti-α-synuclein antibody.

53. The method of any one of claims 29 to 37, wherein the fixed dose is 250 mg of the anti-α-synuclein antibody.

54. The method of any one of claims 29 to 37, wherein the fixed dose is 1250 mg of the anti-α-synuclein antibody.

55. The method of any one of claims 29 to 37, wherein the fixed dose is 3500 mg of the anti-α-synuclein antibody.

56. The method of any one of claims 38 to 49, wherein the method comprises administering intravenously to the human subject a fixed dose of 250 mg of the anti-α-synuclein antibody.

57. The method of any one of claims 38 to 49, wherein the method comprises administering intravenously to the human subject a fixed dose of 1250 mg of the anti-α-synuclein antibody.

58. The method of any one of claims 38 to 49, wherein the method comprises administering intravenously to the human subject a fixed dose of 3500 mg of the anti-α-synuclein antibody.
